(19) United States
(12) Patent Application Publication
(10) Pub. No.: US 2019/0060248 A1
(43) Pub. Date: Feb. 28, 2019

(54) PERCUTANEOUS ABSORPTION PREPARATION

(71) Applicant: Nichiban Co., Ltd., Tokyo (JP)

(72) Inventors: KOJI KAWAHARA, Tokyo (JP); KYOHEI MATSUO, Tokyo (JP); ISAO HAGIWARA, Tokyo (JP); YUKIKO TOHARA, Tokyo (JP); REONA KOIKE, Tokyo (JP)

(21) Appl. No.: 16/087,728

(22) PCT Filed: Mar. 30, 2017
(86) PCT No.: PCT/JP2017/013406

§ 371 Date: Sep. 24, 2018

(30) Foreign Application Priority Data
Mar. 31, 2016 (JP) 2016-071803

(51) Int. Cl. A61K 9/70; A61K 31/167

(52) U.S. Cl. CPC A61K 9/7053; A61K 31/167

(57) ABSTRACT

To provide an adhesive skin patch which achieves both of followability to skin expansion and contraction and a bent face and handleability at the time of pasting.

Provided is a transdermal absorption preparation including an adhesive skin patch and a release liner, in which the adhesive skin patch includes a backing film containing a knitted fabric and/or a nonwoven fabric, an adhesive layer formed on one surface of the backing and containing a pharmacologically active substance, and a carrier film heat-sealed on a surface of the backing film opposite to a surface on which the adhesive layer is formed, and released after the adhesive skin patch is applied to an injured part, the release liner covers at least a part of the adhesive layer of the adhesive skin patch, the carrier film has a bending resistance of 150 mm or less, the backing film has a thickness of 100 μm to 1000 μm, and the carrier film is a film having an area equal to or narrower than the backing film.

### TECHNICAL FIELD

The present invention relates to a transdermal absorption preparation, and it specifically relates to a transdermal absorption preparation, when applied, particularly excellent in handleability at each portion where the preparation is pasted and excellent in followability to the pasted portion.

### BACKGROUND ART

Conventionally, various adhesive skin patches for human have been proposed in medical and health fields. These adhesive skin patches are basically configured by a laminated body formed from a plurality of layers such as an adhesive layer applied to skin, a backing film supporting adhesive layer, and a release layer protecting the adhesive layer until it is applied to skin. The configuration of each layer has been studied and been selected while giving various consideration to intended use, portions to be applied, skin irritation or uncomfortable feeling to applied surfaces (skin), adherability to skin, followability to skin when expanded/contracted, followability to the curved surfaces of skin, and the like.

In particular, in a transdermal absorption preparation (adhesive skin patch) intended for topical action, since an adhesive skin patch is repeatedly applied to a specific injured part, decrease in uncomfortable feeling or physical skin irritation when applied to the injured part will be a major problem. For this reason, as a backing film in the adhesive skin patch, a thinly formed film, a flexible film, a knitted fabric or nonwoven fabric rich in flexibility, and the like have been proposed.

Such a flexible film, a flexibly rich knitted fabric and the like enjoy favorable followability not only to skin expanded/contracted but also the curved surface of skin. On the other hand, they have a defect that handleability would be inferior because of the following. For example, when the release layer is released at the time of application, the adhesive skin patch may be bent so as to make the adhesive layers adhered to each other. Moreover, wrinkles may be generated on the adhesive skin patch when applied to an injured part.

In order to solve such problems, for example, Patent Literature 1 has proposed an adhesive skin patch which achieves improvement in handleability by attaching a specific adhesive planar body to the rear surface of a backing film.

Further, Patent Literature 2 has proposed an adhesive skin patch with a releasable backing film which is laminated on the backing film and has an extension portion extending to the outside of all edges of the backing film. This enables to improve operability at the time of application and to suppress paste protruding to a backing film side.

Furthermore, Patent Literature 3 has proposed an adhesive skin patch which includes a base material having an adhesive surface and a non-adhesive surface and a backing film laminated on the non-adhesive surface of the base material. Patent Literature 3 thus achieves improvement in adaptability at the time of application by which the laminated surface between the non-adhesive surface of the base material and the backing film includes an adhesion part and a non-adhesion part.

### CITATION LIST

### Patent Literatures

Patent Literature 1: JP 10-226638 A.
Patent Literature 2: JP 3158519 U
Patent Literature 3: JP 2010-29242 A

### SUMMARY OF INVENTION

### Technical Problem

As described above, in the backing film of the adhesive skin patch, there have been attempted various propositions which achieve improvement in handleability of the adhesive skin patch in application by employing a configuration in which a carrier layer (corresponding to the planar body in Patent Literature 1, the backing film in Patent Literature 2, or the like) is provided on a surface opposite to a surface on which the adhesive layer is provided. The carrier layer is released after the adhesive skin patch is applied to an injured part.

However, the following problems arise. For example, as disclosed in Patent Literature 1, in a case where an adhesive or a bonding agent is used in order to attach the backing film and the carrier layer to each other until the carrier layer is released from the backing film following the application of the adhesive skin patch, a pharmacologically active substance may be adsorbed into these adhesives and bonding agents. This may lead to deterioration of performance in the transdermal absorption preparation containing a pharmacologically active substance. It would thus become necessary to sufficiently review selection of materials of the carrier layer and the adhesive, a blended composition of the adhesive, and the like, in order to control a release force of the carrier layer from the backing film. Further, since it may be said that the carrier layer itself is configured to include the adhesive layer (for example, the adhesive planar body in Patent Literature 1), the carrier layer itself could be misinterpreted as a drug product.

In addition, in the adhesive skin patch of the Patent Literature 2, in order to provide the extension portion in the releasable backing flu would be necessary to have a process in which a laminated body including the release liner, the adhesive layer, the backing film, and the releasable backing film is configured once, and both ends of the laminated body in a width direction thereof are half-cut to provide slits in only three layers including the release liner, the adhesive layer, and the backing film. The three layers are then removed through the slits so that only the releasable backing film that becomes the extension portion remains. Further, also in the adhesive skin patch of Patent Literature 3, manufacturing would become cumbersome by addition of processes, for example, the process of forming the non-adhesion part.

### Solution to Problem

The present inventors have conducted intensive studies in order to solve the above-described problems and have studied the configuration of a transdermal absorption preparation provided with a carrier layer having rigidity sufficient to improve the handleability of an adhesive skin patch, the carrier layer being provided on the rear surface of the adhesive skin patch which includes a backing film of a knitted fabric or a nonwoven fabric with high flexibility. As a result, the present inventors have found the following. By employing a configuration in which the backing film and the carrier layer are subjected to so-called temporary attachment by heat-sealing, use of the adhesive that may adsorb the pharmacologically active substance can be excluded. In addition, by adjusting a heat-sealing method, a heat-sealing temperature, an area of a heat-sealed portion, a place to be heat-sealed, or the like, a release force of the carrier layer from the backing film (that is, a temporary attachment force between the backing film and the carrier layer) can be more easily controlled than in the related arts. According to this, it has been found that the aforementioned problems such as the manufacturing complication and the performance degradation have been solved, and thus an adhesive skin patch, which satisfies followability to skin when expanded/contracted, followability to curved face of skin and handleability when applied, can be obtained. The present invention has been completed in this manner.

The present invention provides the following transdermal absorption preparation as an embodiment.

(1) A transdermal absorption preparation including an adhesive skin patch and a release liner, in which

the adhesive skin patch includes
a backing film containing a knitted fabric and/or a nonwoven fabric,
an adhesive layer formed on one surface of the backing film and containing a pharmacologically active substance, and
a carrier film heat-sealed on a surface of the backing film opposite to a surface on which the adhesive layer is formed, and released after being applied to an injured part,

the release liner covers at least a part of the adhesive layer of the adhesive skin patch,

the carrier film has a bending resistance of 150 mm or less,

the backing film has a thickness of 100 μm to 1000 μm, and

the carrier film is a film having an area equal to or narrower than the backing film.

Particularly, the present invention provides the following transdermal absorption preparation,

(2) The transdermal absorption preparation described in (1), in which a release force of the carrier film from the backing film is larger than a release force of the release liner from the adhesive layer of the adhesive skin patch.

(3) The transdermal absorption preparation described in (1), in which a release force of the carrier film from the backing film as measured at a release rate of 300 mm/min in a T-shape release test under conditions of 23° C. and 50 RH is 0.05 N/24 mm to 1 N/24 mm.

(4) The transdermal absorption preparation described in (1), in which the carrier film is formed from one or more of thermoplastic resin films.

(5) The transdermal absorption preparation described in (1), in which the carrier film is formed from one or more thermoplastic resin films selected from the group consisting of a cyclic olefin copolymer, polyethylene, polyethylene terephthalate, polypropylene, an ethylene vinyl alcohol copolymer, an ethylene vinyl acetate copolymer, polyvinylidene chloride, and polyacrylonitrile.

(6) The transdermal absorption preparation described in (1), in which carrier film is formed from a film having a half-cut formed thereon.

(7) The transdermal absorption preparation described in (1), in which the carrier film is formed from a heat-sealable polyethylene terephthalate film.

(8) The transdermal absorption preparation described in (1), in which the carrier film is formed from a laminated body obtained by laminating a cyclic olefin copolymer, a polyethylene terephthalate film, and a cyclic olefin copolymer in this order.

(9) The transdermal absorption preparation described in (1), in which the carrier film is formed from a laminated body obtained by laminating a cyclic olefin copolymer and a polyethylene terephthalate film from the backing film side in this order.

(10) The transdermal absorption preparation described in (1) being used for pasting to a shoulder, back, and/or waist.

### Advantageous Effects of Invention

The present invention can provide a transdermal absorption preparation which shows excellent handleability at the time of application by which a carrier film is provided through heat-sealing on a surface of a backing film in an adhesive skin patch opposite to a surface on which an adhesive layer is provided. With this configuration, the present invention enables to easily apply the transdermal absorption preparation to a target place, the carrier film can be easily released from the backing film after the application, and the adhesive skin patch can enjoy excellent followability to a portion applied.

In addition, in the present invention, by controlling a heat-sealing formation between the backing film and the carrier film, for example, by controlling a heat-sealing method, a heat-sealing temperature, the area of a heat-sealed portion, a place to be heat-sealed, the number of the places to be heat-sealed, or the like, the adhesive skin patch can be released from the release liner at the time of using the transdermal absorption preparation. Further, the release strength of the carrier film from the backing film can be easily realized such that the carrier film is not released from the backing film when releasing the adhesive skin patch from the release liner and when the adhesive skin patch is applied, but the carrier film is easily released from the backing film after the application. Further, it is possible to provide a transdermal absorption preparation which achieves improvement in both of handleability at the time of application and followability to an applied portion described above.

### BRIEF DESCRIPTION OF DRAWINGS

FIG. 1 is a cross-sectional view showing one embodiment of a transdermal absorption preparation of the present invention.

### DESCRIPTION OF EMBODIMENTS

The present invention relates to a transdermal absorption preparation including an adhesive skin patch and a release liner.

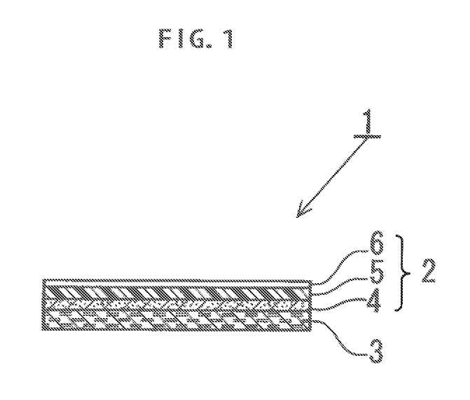

One embodiment of the transdermal absorption preparation of the present invention is illustrated in FIG. 1. As illustrated, a transdermal absorption preparation 1 includes an adhesive skin patch 2 and a release liner 3. The adhesive skin patch 2 includes a backing film 5, an adhesive layer 4 formed on one surface of the backing film 5, and a carrier film 6 that is heat-sealed on a surface of the backing film 5 opposite to a surface on which the adhesive layer 4 is formed.

Hereinafter, the configuration of each layer constituting the transdermal absorption preparation of the present invention will be described in detail.

It is important to variously select the configuration of each of these layers while considering adherability to an application target surface (skin), operability of the adhesive skin patch, and the like.

### Adhesive Skin Patch

In the transdermal absorption preparation of the present invention, the adhesive skin patch has a laminate structure of at least three layers (a carrier film, a backing film, and an adhesive layer) including a backing film containing a knitted fabric and/or a nonwoven fabric, an adhesive layer formed on one surface of the backing film and containing a pharmacologically active substance, and a carrier film heat-sealed on a surface of the backing film opposite to a surface on which the adhesive layer is formed+and released after being applied to an injured part.

### Backing Film

In the present invention, the backing film contains a knitted fabric or a nonwoven fabric, or both a knitted fabric and a nonwoven fabric.

Based on the assumption that the backing film is a material which can be heat-welded to the carrier film to be described below (heat-sealable material), the backing film would be preferable when satisfying: a flexible material with such a degree of flexibility that the backing film can adhere to skin and can follow skin movement; and a material which can suppress the occurrence of skin rash or the like even after being applied for a long period of time. In particular, from the viewpoint of excellent followability to skin movement during the application (expanded/contracted skin portions or bent skin faces), for example, a knitted fabric or a nonwoven fabric formed from fibers of a resin of polyester such as polyethylene terephthalate or polybutylene terephthalate, polyolefin such as polyethylene or polypropylene, polyurethane, or the like is preferable. Among them, a polyester knitted fabric is suitable.

Incidentally, as the backing film, in addition to the knitted fabric or/and the nonwoven fabric, in a range that the followability to the skin movement is not damaged, a backing film formed in a laminate structure using a backing film material, for example, paper such as impregnated paper, coated paper, high-quality paper, kraft paper, Japanese paper, or glassine paper; a plastic film such as polyester, polyolefin, polyurethane, polyvinyl polycarbonate, or cellophane; or a foam may be used.

The thickness, total weight, and bending resistance of the backing film are set in consideration of physical properties such as elongation, tensile strength, and workability, feeling when applied, sealability to an injured portion, influence on transition of each component (for example, a pharmacologically active substance or the like) contained in the adhesive layer described later to the backing film or stability of the pharmacologically active substance, and the like.

In the present invention, the thickness of the backing film is set n a range of 100 μm to 1000 μm. The thickness of the backing film is preferably 200 μm to 800 μm, more preferably 300 μm to 700 μm, and further preferably 400 μm to 600 μm. In addition, the total weight of the backing film is preferably 300 g/m² or less, more preferably 200 g/m² or less, and further preferably 150 g/m² or less in terms of followability to skin. The lower limit of the total weight of the backing film is 50 g/m² or more and preferably 75 g/m² or more. Further, the bending resistance of the backing film is preferably 8 mm to 30 mm and more preferably 10 mm to 18 mm in terms of followability to skin.

When the thickness of the backing film is smaller (thinner) than the above numerical range or the total weight thereof is smaller than the above numerical range, the strength and handleability of the backing film are decreased so that application to skin would become difficult even when the carrier film is provided. Thus, the backing film may be torn due to contact with another member or the like. Further, the backing film may be torn at skin expansion and contraction portions or skin bent faces. Still further, the backing film may be released from skin even within a short period of time due to contact with water in a bath or the like. In addition, when the thickness of the backing film is excessively large (more than 1 mm) or the total weight thereof exceeds the above numerical range, the backing film (and eventually the adhesive skin patch) is less likely to follow skin movement and is likely released from the periphery of the adhesive skin patch. Thus, the adhesive skin patch may be released from skin within a short period of time, or uncomfortable feeling during application may increase.

Further, in order to secure flexibility that enables to follow the expansion and contraction portions or curved faces of the skin and to secure adhesiveness to skin, for example, the tensile strength at 20% modulus of the backing film is preferably set to 1 N/25 mm or less, and the tensile strength at 50% modulus thereof is preferably set to 10 N/25 mm or less.

Furthermore, it is desirable that the pharmacologically active substance contained in the adhesive layer is not adsorbed to the backing film, and the pharmacologically active substance is not discharged from the backing film side.

Incidentally, in order to make the adhesive skin patch inconspicuous when applied to skin, that is, in order to reduce a difference between the adhesive skin patch and skin color when applied, the backing film may be colored to have a color tone such as skin color with colorant, for example, a pigment, an organic pigment, or a natural pigment.

Further, the backing film may contain an additive such as an antistatic agent or an anti-ultraviolet agent to such a degree that the effect of the present invention is not damaged. Examples of the antistatic agent include surfactants (such as an anionic surfactant, a cationic surfactant, a non-ionic surfactant, and an ampholytic surfactant)

### Adhesive Layer

The adhesive layer in the adhesive skin patch of the present invention contains an adhesive and a pharmacologically active substance as essential constituents.

The adhesive is not particularly limited, and the examples thereof may include an acrylic adhesive, a rubber-based adhesive, a urethane-based adhesive, and a silicone-based adhesive, and the like. These adhesives may be used alone or as a mixture of two or more kinds thereof. Among them, from the viewpoint of compatibility with a blended component, or the like, a rubber-based adhesive is suitably used.

The rubber-based adhesive typically contains a rubber-based elastomer, a tackifier, and a softener, and as necessary, is further added with various additives such as a filler and an antioxidant (oxidation inhibitor) described later.

As the rubber-based elastomer, it is possible to employ various thermoplastic elastomers, for example, thermoplastic block copolymers such as a styrene-isoprene-styrene copolymer (hereinafter, referred to as “SIS” when necessary), a styrene-butadiene-styrene copolymer (hereinafter, referred to as “SBS” when necessary), or hydrogenated products thereof, a styrene-ethylene-propylene-styrene copolymer (hereinafter, referred to as “SEPS” when necessary), and a styrene-ethylene-butylene-styrene copolymer (hereinafter, referred to as “SEBS” when necessary); ethylene-vinyl acetate copolymers; and ethylene-α-olefin copolymers. Among these, styrene-based thermoplastic elastomers that are thermoplastic block copolymers, such as SIS, SBS, SEPS, and SEBS, are suitably used from the viewpoint of having excellent adhesion and cohesiveness. Of these, from the viewpoint of adhesive power to human skin, compatibility with another component, or the like, SIS is particularly preferable. The content of SIS is not particularly limited, and is preferably 10% by mass to 50% by mass and more preferably 10% by mass to 40% by mass when the total mass of the adhesive layer is 100% by mass. As SIS, a commercially available styrene-isoprene-styrene block copolymer can be used, and for example, JSR SIS 5002 (JSR Corporation) can be exemplified.

Examples of the tackifier include a rosin-based resin, a terpene-based resin, a coumarone-indene resin, a petroleum-based resin (a C5-based petroleum resin or a C9-based petroleum resin), an alicyclic petroleum resin, an alicyclic hydrogenated petroleum resin, a styrene-based resin, and a dicyclopentadiene resin and the like. The content of the tackifier is not particularly limited, and for example, can be set to preferably 15% by mass to 55% by mass and more preferably 20% by mass to 50% by mass when the total mass of the adhesive layer is 100% by mass.

Examples of the softener (plasticizer) include a petroleum-based softener such as liquid paraffin; a liquid rubber-based softener such as liquid polyisoprene, polybutene, or polyisobutylene; a dibasic acid ester-based plasticizer such as phthalic acid ester or adipic acid ester; and another plasticizer such as polyethyleneglycol or citric acid ester. Among them, since liquid paraffin is excellent in compatibility with a rubber-based elastomer system and there is no concern that the cohesive power thereof is decreased, liquid paraffin can be preferably used, and examples of commercially available liquid paraffin include Hicall (registered trademark, liquid paraffin manufactured by KANEDA Co., Ltd.) M series. In terms of adhesion, the content of these softeners is preferably in a range of 25% by mass to 55% by mass and more preferably 30% by mass to 50% by mass when the total mass of the adhesive layer is 100% by mass.

The rubber-based adhesive can further contain an additive, which is typically blended in the adhesive layer of the transdermal absorption preparation, such as an antioxidant (antioxidant agent), a filler, a transdermal absorption promotor, a pigment, a stabilizing agent, a solubility improver, or a solubility inhibitor, as necessary. These additives can be used alone or in combination of two or more kinds thereof.

The pharmacologically active substance contained in the adhesive layer of the adhesive skin patch is not particularly limited, and examples of a systemic pharmacologically active substance include corticosteroids, analgesic/anti-inflammatory agents, hypnotic/analgesic agents, tranquilizers, antihypertensives, antihypertensive diuretics, antibiotics, general anesthetics, antibacterial agents, antimycotic agents, vitamins, coronary vasodilators, antihistamine agents, antitussive agents, sex hormones, antidepressant agents, cerebral circulation activators, antiemetic agents, antitumor agents, enzyme agents, and biopharmaceuticals.

Among them, as the suitable pharmacologically active substance intended for the transdermal absorption preparation of the present invention, for example, lidocaine that is a regional anesthetic can be exemplified. As the lidocaine, lidocaine or a pharmaceutically acceptable salt (lidocaine hydrochloride or the like) can be used. These may be contained alone or as a mixture, but it is preferable that lidocaine is contained alone in order for lidocaine to be contained in a dissolved state in the adhesive layer.

The blended amount of the pharmacologically active substance based on the total mass of the adhesive layer is appropriately selected, for example, in a range of 0.1% by mass to 50% by mass depending on the kind of pharmacologically active substance to be blended, application conditions of the transdermal absorption preparation (such as application frequency or application time), and the like. In the case of blending the above-described lidocaine, the blended amount thereof can be appropriately selected within a range of preferably 1.5% by mass to 6.5% by mass and more preferably 3.0% by mass to 6.0% by mass.

The thickness of the adhesive layer is not particularly limited, but can be set, for example, in a range of 5 μm to 500 μm, preferably 10 μm to 400 μm, more preferably 35 μm to 300 μm, and further preferably 40 μm to 200 μm.

### Carrier Film

In the adhesive skin patch, the carrier film is heat-sealed on a surface of the backing film opposite to a surface on which the adhesive layer is provided. The carrier film is provided to improve handleability of the adhesive skin patch and has higher stiffness than the backing film.

Therefore, the carrier film may cover the entire surface or a part of the adhesive skin patch (backing film). For example, the carrier film may cover only the edge of the adhesive skin patch (backing film) or may cover the adhesive skin patch in a pattern shape such as a lattice shape. That is, the carrier film can be a film having an area equal to or narrower (small dimension) than the backing film.

Further, a half-cut may also be provided in the carrier film to facilitate release from the backing film.

Since the backing film is temporarily attached to the carrier film by heat-sealing, handleability of the adhesive skin patch and application properties to an adherend can be improved.

It is desirable that the carrier film is increased in thickness or is formed from a rigid material for improving handleability of an adhesive tape. The thickness of the carrier film is typically 10 μm to 500 μm, preferably 20 μm to 250 μm, and particularly preferably 30 μm to 100 μm. When the thickness of the carrier film is less than 10 μm, the backing film of the adhesive tape and the carrier film do not sufficiently adhere to each other. In addition, when the thickness thereof is more than 500 μm, adhesiveness to the backing film of the adhesive tape will be sufficient and operability will be also improved. However, stiffness of the carrier film becomes excessively high. For example, when the release liner is released and the adhesive layer is then applied to skin in use, followability to skin becomes deficient and application properties to curved portions or the like will be insufficient. Further, if the thickness of the carrier film is equal to or larger than the thickness of the backing film, there is a concern that a sense of unity between the backing film and the carrier film will become deficient when releasing the adhesive skin patch from the release liner. This thus causes only the carrier film to be released from the adhesive skin patch. For this reason, it is desirable that the carrier film has a thickness equal to or less than the backing film. The rigidity of the carrier film is definable based on bending resistance, and the bending resistance of the carrier film may be 150 mm or less and preferably 40 mm or more and 90 min or less.

As the carrier film, for example, various films formed from various thermoplastic resins such as polyester (polyethylene terephthalate or the like), polyurethane, polyolefin (polyethylene, polypropylene or the like), ionomer, polyamide, polyvinyl chloride, polyvinylidene chloride, an ethylene vinyl acetate copolymer, thermoplastic polyester, and polytetrafluoroethylene are usable. Those obtained by laminating the various films on paper may also be used.

Among them, as the carrier film, it is preferable to employ those formed from one or more thermoplastic resin films selected from the group consisting of a cyclic olefin copolymer (hereinafter, abbreviated as “COC” when necessary), polyethylene, polyethylene terephthalate (hereinafter, abbreviated as “PET” when necessary), polypropylene, an ethylene vinyl alcohol copolymer, an ethylene vinyl acetate copolymer, polyvinylidene chloride, and polyacrylonitrile. Especially, from the viewpoint of favorable handleability of the adhesive skin patch, a heat-sealable polyethylene terephthalate film (hereinafter, abbreviated as “hs-PET” when necessary) or a laminated body of COC and PET is preferably used.

Further, since the carrier film is provided to improve handleability of the adhesive skin patch, when storing the transdermal absorption preparation or before applying the adhesive skin patch to skin (or when applying the adhesive skin patch to skin), the carrier film should not be released from the backing film, and at least a part thereof should be kept temporal attachment. Therefore, it is desirable that when the release liner is released from the adhesive layer of the adhesive skin patch at the time of using the transdermal absorption preparation, the carrier film is not yet released from the backing film. Specifically, it is desirable that a release force of the carrier film from the backing film is larger than a release force of the release liner from the adhesive layer of the adhesive skin patch.

For example, regarding the release force of the carrier film from the backing film, a release force as measured at a release rate of 300 min/min in a T-shape release test under conditions of 23° C. and 50 RH can be set to 0.05 N/24 mm to 1 N/24 mm.

In the present invention, when the carrier film is heat-sealed to the backing film, the carrier film and the backing film will be in a state of so-called temporary attachment. On the other hand, the backing film and the adhesive layer will be kept a firm attachment through the adhesive contained in the adhesive layer. Accordingly, ease of release of the carrier film from the backing film will be available after the adhesive skin patch has been applied.

Further, in the present invention, since the carrier film and the backing film are temporarily attached by heat-sealing without using an adhesive or a bonding agent, a concern of adsorption of the pharmacologically active substance by an adhesive or a bonding agent, which has been conventionally used for the attachment between the carrier film (carrier layer) and the backing film, can be suppressed.

Examples of a particularly preferable carrier film include a heat-sealable polyethylene terephthalate film (hs-PET), a laminated body in which cyclic polyolefin and polyethylene terephthalate are laminated from the backing film side in this order (hereinafter, abbreviated as “COC/PET” when necessary), and a laminated body in which cyclic polyolefin, polyethylene terephthalate, and cyclic polyolefin are laminated in this order (hereinafter, abbreviated as “COC/PET/COC” when necessary), as described above. These laminated bodies (COC/PET or COC/PET/COC) are laminated through an adhesive or the like. At this time, it is desirable to use an adhesive to which the pharmacologically active substance is less adsorbed. These hs-PET and laminated bodies are almost transparent. Thus, it can be easily confirmed during processes whether the backing film and the carrier layer are well heat-sealed. In addition, handleability of the adhesive skin patch after releasing the release liner will be notably easy for a user. Adsorption of the pharmacologically active substance can also be reduced.

In particular, when the carrier film is configured by the laminated body of COC/PET or the laminated body of COC/PET/COC, a welding temperature when temporary attachment is performed by heat-welding can be set to be lower than that of the heat-sealable polyethylene terephthalate film. Thus, excessive heat to the backing film will be avoidable when temporarily attaching the carrier layer and backing film by heat-welding, which is preferable. Regarding the welding temperature when the temporary attachment is performed by the heat-welding, the heat-welding is performed preferably at 140° C. to 150° C. in the case of the configuration of the laminated body of COC/PET or COC/PET/COC and at 160° C. to 200° C. in the case of the heat-sealable polyethylene terephthalate film.

Further, when the carrier film is configured by the laminated body of COC/PET or the laminated body of COC/PET/COC, the adsorbed amount of the pharmacologically active substance to the carrier film can be decreased as compared to the heat-sealable polyethylene terephthalate film, which is preferable.

Furthermore, in the laminated body of COC/PET and the laminated body of COC/PET/COC, the thickness of each of COC and PET can be appropriately adjusted. It is preferable that a ratio of thickness of the PET layer to each layer of COC is COC: PET=10 to 100:10 to 50 and the thickness of COC is thicker than that of PET, and more preferable that the ratio of thickness is COC: PET=10 to 50:10 to 30 and the thickness of COC is thicker than that of PET, which is preferable in terms of excellent heat-sealing performance.

Further, COC (cyclic olefin copolymer) used in the carrier film may be mixed with an olefin-based resin such as a linear low-density polyethylene resin (LLDPE), a high-density polyethylene resin (HDPE), or a polypropylene resin (PP) in consideration of adhesion properties (heat-sealing properties), an eluted amount of low-molecular weight, flowability, or the like. In the present invention, a blend product with such an olefin-based resin can also be included in “COC.” In this case, a blend rate (mass ratio) of an olefin-based resin other than COC constituting the COC film is preferably 3% by mass to 50% by mass and particularly preferably 5% by mass to 10% by mass. When the blend ratio of the olefin-based resin to the total mass of the blend product is less than 3% by mass, appropriate flowability cannot be applied to the cyclic polyolefin-based resin, and this causes gel blocks to be generated. On the other hand, when the blend ratio of the olefin-based resin is more than 50% by mass, non-adsorbing properties of cyclic polyolefin may deteriorate. In addition, transparency may be degraded although not directly influencing on the effect of the present invention.

Incidentally, the density (g/cm³) of the linear low-density polyethylene resin is 0.935 to 0.950, and the density (g/cm³) of the high-density polyethylene resin is 0.940 to 0.975. The polypropylene resin may be either homo-type or block-type, but a homo-type polypropylene resin is preferable.

In addition, if necessary, one or more kinds of additives such as an antioxidant, an ultraviolet absorber, a light stabilizer, an antistatic agent, an antiblocking agent, a lubricant (fatty acid amide or the like), a flame retardant, an inorganic or organic filler, a cross-linking agent, a dye, a colorant such as a pigment, and a modifying resin may be further contained.

As a polymer constituting the COC film, examples of a commercially available product which can be used include TOPAS (registered trademark, manufactured by Polyplastics Co., Ltd.), APEL (registered trademark, manufactured by Mitsui Chemicals, Inc.), and ARTON (registered trademark, manufactured by JSR Corporation). As the COC film, examples of a commercially available product which can be used include ZeonorFilm (registered trademark, manufactured by Zeon Corporation).

Further, in the COC film, a structural unit derived from an olefin component such as ethylene is appropriately in a range of 40 mol % to 95 mol %, and a structural unit derived from a cyclic olefin component is typically appropriately in a range of 5 mol % to 60 mol %.

The release force (release strength) of the carrier film from the backing film described above can be adjusted by controlling the conditions of heat-sealing, for example, a heat-sealing method, a heat-sealing temperature, an area of a heat-sealed portion, a place to be heat-sealed, the number of the places when performing heat-sealing at a plurality of places, and the like.

For example, in the heat-sealing, methods typically adopted in conventional heat-sealing processes, such as heat press (pattern roll or hot plate), laser melting, hot air, and infrared irradiation may be employed. In any of the methods, a heat-sealing temperature (heat quantity) should be adjustable. Further, the heat-sealing method and the heat-sealing temperature can be appropriately selected depending on materials/thickness of the backing film and the carrier film, or an intended release strength (temporary adhesive strength).

The heat-sealing of the backing film and the carrier film can be performed, for example, over the entire surface or a predetermined portion of the carrier film in the adhesive skin patch. In addition, the shape of the heat-sealed portion can be a point shape, a plane shape such as a circle or a polygon, or a shape combined thereof.

For example, as the heat-sealing method, hot press may be taken so that the backing film and the carrier film are heat-sealed to be a pattern shape such as a dot shape, a line shape, or a mesh shape, for example, at a temperature of 100° C. to 200° C.

Further, in the carrier film, a method of using the transdermal absorption preparation of the present invention, for example, a release procedure and a release method of the release liner or the carrier film, the kind of the adhesive kin patch (the kind of active ingredient to be blended), and the like can be defined on the surface of the backing film opposite to a heat-sealed surface by means of printing, embossing, or the like.

In a manufacturing process for the transdermal absorption preparation described below, for example, a laminated body configured by the carrier film/the backing film/the adhesive layer/the release liner is produced and then the laminated body is wound in a roll shape and stored until the transdermal absorption preparation is cut in a desired shape. However, when the laminated body is stored as the roll laminated body, wrinkle (deflection) may be generated on the carrier film that is temporarily attached to the backing film. This is why the shape of heat-sealing between the backing film and the carrier film should be controlled, for example, by making the shape to be a point shape (point adhesion), it can be expected that the generation of wrinkle on the carrier film in the laminated roll body is prevented.

In the transdermal absorption preparation of the present invention, a support liner may be pasted to the carrier film in order to facilitate release of the carrier film from the backing film after the adhesive skin patch has been applied. The support liner is configurable, for example, by a tape backing layer and a support adhesive layer However, since the backing layer is sufficiently thick in general, the carrier film should not be difficult to be released, and handleability of the transdermal absorption preparation of the present invention should be favorable. Therefore, the support liner will be optional.

### Shape of Adhesive Skin Patch

The shape of the adhesive skin patch is not particularly limited, and various shapes such as a quadrate (square, rectangle, or the like), a tetragon (trapezoid, rhombus, or the like), a polygon, a circle, an ellipse, a semicircle, a triangle, a crescent, and a shape combined thereof, may be selected in consideration of portions where the adhesive skin patch is applied.

Incidentally, the size of the adhesive skin patch may be appropriately determined, for example, it can be set in a range of 2 cm² to 300 cm² in consideration of the dosage purpose and dosage amount of the pharmacologically active substance. In the case of a lidocaine-containing adhesive skin patch, the size of the adhesive skin patch may be set to 40 cm² to 240 cm².

### Release Liner

The release liner (also referred to as release layer or release paper) used in the adhesive skin patch of the present invention is released when used. This release liner is provided for protecting a layer (adhesive layer) that meets skin until being used, so that quality deterioration can be prevented. In the present invention, the adhesive skin patch indicates a lamination including: the backing film; the adhesive layer containing a pharmacologically active substance; and the carrier film to be released after applied to an injured part. The transdermal absorption preparation indicates a lamination defined by the release liner provided on the adhesive surface of the adhesive skin patch (a surface of the adhesive layer opposite to a side at which the backing film is provided). As the release liner, a release liner common in the technical fields of transdermal absorption preparations and adhesive products (adhesive skin patches) may be used while considering some influences of transition of each component contained in the adhesive layer (for example, the pharmacologically active substance or the like) to the release liner, stabilities of the pharmacologically active substance, or the like. Examples of the release liner may include colorless or colored sheets such as plastic films of polyester (polyethylene terephthalate, polybutylene terephthalate, polyethylene naphthalate, or the like), polypropylene (unstretchable or stretchable, etc.), polyethylene, polyurethane, polyvinyl chloride, polystyrene, and the like; paper or synthetic paper such as high-quality paper, glassine paper, parchment paper, kraft paper, and the like; release-processed paper obtained by coating a release agent having release performance, such as a silicone resin or a fluororesin, on the plastic film, the paper or synthetic paper, synthetic fibers, or the like; aluminum foil; laminate-processed paper obtained by variously laminating these film and sheets, and laminate-release-processed paper obtained by coating a release agent on the laminate-processed paper.

Among these, in consideration of suppression of adsorption of the pharmacologically active substance and the handleability of the transdermal absorption preparation until the transdermal absorption preparation is taken out from a package and then the release liner is released, polyethylene terephthalate is preferable. A polyethylene terephthalate film used in the release liner is different from heat-sealable polyethylene terephthalate illustrated in the carrier film and indicates polyethylene terephthalate having a melting point of near 250° C.

When focusing on stiffness of respective layers in the transdermal absorption preparation (magnitude of bending resistance), it is preferable to satisfy the following when handleability is considered: the release liner>the carrier film>the adhesive skin patch. For example, when using bending resistance as an index, the lending resistance would be particularly preferable when satisfying that the release liner: the carrier film the adhesive skin patch (or the backing film)=100 mm to 150 mm: 40 mm to 90 mm: 8 mm to 30 mm, and the release liner>the carrier film>the adhesive skin patch (or the backing film).

The thicknesses of the release liner is not particularly limited, and is in a range of typically 10 μm to 1 mm, for example, 20 μm to 500 μm, and preferably 40 μm to 200 μm. In order to obtain appropriate stiffness, the thickness thereof is particularly preferably 50 μm to 150 μm. In addition, the bending resistance of the release liner is preferably 100 mm or more, and further preferably 110 mm to 150 mm in terms of handleability.

In addition, the shape of the release liner may be a square, a circle, or the like, and may be a shape obtained by rounding a corner (R shape) as desired. The size thereof may be the same as or slightly larger than the size of the backing film in the adhesive skin patch. The release liner may be configured as one sheet or may be divided into a plurality of sheets. The cut line thereof may be formed in a straight line, a wavy line, a perforated line, or the like. Parts of the release liners may be superimposed to each other.

<Method for Manufacturing Transdermal Absorption Preparation>

A method for manufacturing the transdermal absorption preparation intended by the present invention is not particularly limited, and conventional methods typically performed in the transdermal absorption preparation and the adhesive tape may be suitably combined and be adopted. Incidentally, in one of the processes in which to manufacture the transdermal absorption preparation, it would be preferable to include a process of temporarily attaching the carrier film and the backing film of the adhesive skin patch by heat-sealing thereby producing the laminate structure being composed of the carrier film and the backing film of the adhesive skin patch.

In a preferred embodiment in the method for manufacturing the transdermal absorption preparation of the present invention, first, the carrier film and the backing film of the adhesive skin patch are heat-sealed to produce a laminated body of the backing film of the adhesive skin patch and the carrier film. The heat-sealing method, the heat-sealing temperature, an area of the heat-sealed portion, a place to be heat-sealed, and the number of the places to be heat-sealed can be appropriately set as described above.

Next, additionally, the adhesive layer is formed on the release liner by applying a material for forming the adhesive layer of the adhesive skin patch containing an adhesive and a pharmacologically active substance to the release liner. Thereafter, the adhesive layer is pasted with the backing film surface of the laminated body, and the obtained product is cut from the carrier film side so that the transdermal absorption preparation in which the adhesive skin patch is pasted onto the release liner can be obtained. In general, the transdermal absorption preparation is then sealed in an appropriate package to be stored.

As a method for forming the adhesive layer, a hot-melt method, a calender method, a spread coating method, an emulsion method, an electron beam curing method, and the like that are methods for conventionally forming the adhesive layer can be adopted in consideration of the kind of the adhesive contained in the adhesive layer, the kind of the pharmacologically active substance, and the like. For example, in the case of forming an adhesive layer containing lidocaine, a manufacturing method in which moisture is not intentionally added to the adhesive layer (plaster) in the manufacturing process will be preferably adopted.

### Method of Using Transdermal Absorption Preparation

In the method of using the transdermal absorption preparation of the present invention, first, the transdermal absorption preparation is taken out from a package generally used, the release liner of the transdermal absorption preparation is released, the adhesive surface of the adhesive skin patch is pasted to an application portion, and the carrier film is then released. Through the procedures, the pasting of a member configured by the backing film and the adhesive to skin is completed. Because of the carrier film to be provided, stiffness of the backing film configured by a nonwoven fabric or a knitted fabric is increased. Thus, the transdermal absorption preparation of the present invention can be handled by oneself even at a location that is difficult to directly check by eyes, for example, a shoulder, back, and/or waist.

### EXAMPLES

Hereinafter, the present invention will be described in detail by means of examples and comparative examples. However, the present invention is not limited to these examples and various applications are possible as long as they do not deviate from the technical idea of the present invention.

Measurement methods of characteristics and physical properties of respective layers of the transdermal absorption preparation of the present invention are as follows.

### Thickness

The thicknesses of the carrier film, the backing, the adhesive layer, and the release liner were measured using a dial thickness gauge.

### Example 1

In the hot-melt method described in Method for Manufacturing Transdermal Absorption Preparation, 3.0% by mass of lidocaine as a pharmacologically active substance, 32.0% by mass of a styrene-isoprene-styrene block copolymer [JSR SIS 5002 manufactured by JSR Corporation], 17.5% by mass of hydrogenated rosin glycerin ester [PINECRYSTAL KE-311 manufactured by ARAKAWA CHEMICAL INDUSTRIES, LTD.] and 10.0% by mass of a terpene resin [YS Resin PX1150N manufactured by YASUHARA CHEMICAL CO., LTD] as tackifier resins, and 37.5% by mass of liquid paraffin [Hicall (registered trademark) M-352 manufactured by KANEDA Co., Ltd.] were blended (numerical values (% by mass) indicate numerical values when the total mass of the adhesive layer is 100% by mass, the same applies hereinafter) and heated under stirring to prepare a homogeneous adhesive composition. Incidentally, the heating under stirring was performed in such a manner that the above-described materials other than the pharmacologically active substance were melted under stirring in a Henschel mixer under a nitrogen atmosphere so as to be in a homogeneous state.

Next, the adhesive composition was spread on a polyester film subjected to a silicone treatment (thickness: 75 μm) to have a thickness of 200 g/m², thereby producing the adhesive layer. Through this, a laminated body A including the release liner and the adhesive layer was obtained.

Meanwhile, a polyester knitted fabric (circular knitting, total weight: about 100 g/m², thickness: about 500 μm) as the backing film and a heat-sealable polyethylene terephthalate film (hs-PET: thickness of 40 μm) as the carrier film were temporarily attached to the entire surface of the backing film by heat-welding (160° C.), thereby obtaining a laminated body B including the backing film and the carrier film.

Finally, the laminated body A and the laminated body B were pasted to each other such that the adhesive layer of the laminated body A and the backing film of the laminated body B were superimposed. Thereafter, the obtained product was cut to have a shape of 10 cm×14 cm to produce the transdermal absorption preparation of Example 1 (see FIG. 1).

In the transdermal absorption preparation thus produced, the adhesive skin patch was released from the release liner, the adhesive layer was pasted to a waist of a person, and then the carrier film could be released. Thus, favorable handleability of the transdermal absorption preparation was confirmed.

Hereinafter, transdermal absorption preparations of examples and comparative examples were obtained by which the configuration of the carrier film is variously changed.

### Example 2

A transdermal absorption preparation of Example 2 was obtained in a similar manner to Example 1, except that a laminated body (total thickness: 45 μ) of COC/PET, which is placed from the backing film side, was used as the carrier film instead of hs-PET, and the heat-welding temperature of the backing film and the carrier film was accordingly set to 146° C. Incidentally, the details of the carrier film used in this example are as follows.

Carrier film: COC film (thickness: 30 μm)/adhesive/PET film (thickness: 12 μm)

### Example 3

A transdermal absorption preparation of Example 3 was obtained in a similar manner to Example 1, except that a laminated body (total thickness: 78 μm) of COC/PET/COC was used as the carrier film instead of hs-PET and the heat-welding temperature of the backing film and the carrier film was accordingly set to 146° C. Incidentally, the details of the carrier film used in this example are as follows.

Carrier film: COC film (thickness: 30 μm)/adhesive/PET film (thickness: 12 μm)/adhesive/COC film (thickness: 30 μm)

### Comparative Example 1

A transdermal absorption preparation of Comparative Example 1 was obtained in a similar manner to Example 1, except that the carrier film was not provided.

### Comparative Example 2

A transdermal absorption preparation of Comparative Example 2 was obtained in a similar manner to Example 1, except that a heat-sealable polyethylene terephthalate film (thickness: 150 μm) having a different thickness was used as the carrier film

### Evaluation for Handleability

Regarding the ease of release of the release transdermal absorption preparation and the ease of pasting the adhesive skin patch to waist, sensory evaluation was performed based on body sensations of a testee. In addition, the bending resistance of each of the backing film, the carrier film, and the release liner were measured. Results thereof are presented in Table 1.

### Sensory Evaluation by Body Sensations of Testee

The evaluation method is as follows.

Testee: Seven people

Grade average: [Evaluation result (0 point to 100 points): addition of each evaluation of seven people] divided by Number of people (seven people)

### Ease of Release of Release Liner

Release is very easy: 100 points

Release is relatively easy: 75 points

Release is neither easy nor difficult: 50 points

Release is slightly difficult: 25 points

Release is very difficult: 0 point

### Ease of Pasting

Pasting is very easy: 100 points

Pasting is relatively easy: 75 points

Pasting is neither easy nor difficult: 50 points

Pasting is slightly difficult: 25 points

Pasting is very difficult and problematic: 0 point

### Bending Resistance

Respective samples of the backing film, the carrier film, and the release liner used in the transdermal absorption preparations of examples and comparative examples were cut into six pieces with each size of a vertical direction×a horizontal direction=150 mm×20 mm and a horizontal direction×a vertical direction=150 mm×20 mm to produce test pieces (12 pieces for each). The bending resistance at each of front and rear sides of the test piece was measured using a cantilever-type bending resistance tester (45°) and an average value was calculated.

TABLE 1

| | | | Sensory evaluation | | Bending resistance | | |
|---|---|---|---|---|---|---|---|
| | Carrier film | | (average point) | | (mm) | | |
| | | Thickness | Ease of Release | Ease of | Carrier | Backing | Release |
| | Material | (μm) | of release liner | pasting | film | film | liner |
| Example 1 | hs-PET | 40 | 82 | 79 | 59 | 14 | 113 |
| Example 2 | COC/PET | 45 | 80 | 84 | 64 | 14 | 113 |
| Example 3 | COC/PET/COC | 78 | 78 | 89 | 79 | 14 | 113 |
| Comparative | — | — | 83 | 65 | — | 14 | 113 |
| Example 1 | | | | | | | |
| Comparative | hs-PET | 150 | 68 | 93 | 150< | 14 | 113 |
| Example 2 | | | | | | | |

Test Result and Study thereto

As presented in Table 1, it was found that Example 1 to Example 3 using the carrier film are easy-to-use transdermal absorption preparations in which the ease of release of the release liner from the transdermal absorption preparation and the ease of pasting the adhesive skin patch to waist are favorable. On the other hand, in Comparative Example 1 with no carrier film, the ease of pasting the adhesive skin patch to waist was not favorable. The reason for this was considered as follows. Since the bending resistance of the backing film is as small as 14 mm and the adhesive skin patch is bent, adhesive faces adhere to each other. This will make pasting difficult. In addition, in Comparative Example 2 using the carrier film of hs-PET (thickness: 150 μm), the ease of release of the release liner from the transdermal absorption preparation was not favorable. The reason for this was considered as follows. Since the bending resistance of hs-PET (thickness: 150 μm) is larger than that of the release liner and the rigidity of the carrier film is large, this will make the release liner difficult to be released.

### Evaluation on Release Force of Carrier Film

By using the laminated body B prepared in each of Example 1, Example 2, and Example 3, the release force of the carrier film from the backing film was measured. Then, a relation between a welding temperature of the carrier film to the backing film and the release force was evaluated. The release force of the carrier film was measured at a release rate of 300 mm/min in a T-shape release test under conditions of 23° C. and 50% RH while the transderma absorption preparation was cut into a width of 24 mm. The results obtained are presented in Table 2.

TABLE 2

| | Carrier film | | Welding | |
|---|---|---|---|---|
| | | Thickness | temperature | Release force |
| | Material | (μm) | (° C.) | (N/24 mm) |
| Example 1 | hs-PET | 40 | 160 | 0.24 |
| Example 2 | COC/PET | 45 | 146 | 0.24 |
| Example 3 | COC/PET/COC | 78 | 146 | 0.24 |

Test Result and Study thereto

As presented in Table 2, in Example 1, the release force of the carrier film was 0.24 N/24 mm at a welding temperature of 160° C., and in Example 2 and Example 3, the release force of the carrier film was 0.24 N/24 mm at a welding temperature of 146° C. The favorable release force was obtained at a lower welding temperature in the COC film rather than the hs-PET film. As a result, it was considered that when the welding temperature is low, this could make the heat-welding rate further accelerated so that production efficiency could be improved in the COC film rather than the hs-PET film. In addition, it was considered that when the welding temperature is low, heat-welding temperature unevenness could be reduced so that the accuracy of the release force of the carrier film is enhanced in the COC film rather than the hs-PET film.

### Evaluation on Adsorbed Amount of Pharmacologically Active Substance to Carrier Film

When the transdermal absorption preparations produced in Example 1, Example 2, and Example 3 were sealed in an aluminum package and stored under a severe condition (60° C.) for 14 days, the adsorbed amount of lidocaine to the carrier film was measured according to the following [Content and Measurement Procedure]. The adsorbed amount of lidocaine to the carrier film was then evaluated, and Table 3 shows the results obtained.

### Content and Measurement Procedure

The transdermal absorption preparation of each example was taken out from the aluminum package after being stored under the severe condition, and the carrier film was released. Then, the carrier film was immersed in a sealable glass container including an internal standard solution and tetrahydrofuran for HPLC. Thereafter, a mixed solution of acetonitrile for a drug/sodium dihydrogen phosphate buffer solution (pH 3) was added thereto to prepare a sample solution.

Separately, by using a lidocaine standard product, a standard solution was prepared by a similar operation.

The sample solution and the standard solution were analyzed by a high-performance liquid chromatography (HPLC) method. The amount of lidocaine in the sample (the adsorbed amount of lidocaine to the carrier film) was calculated from a ratio of a peak area of lidocaine to a peak area of an internal standard substance in each of the standard solution and the sample solution.

From the obtained results, the adsorbed amount (%) of lidocaine to the carrier film was calculated on the basis of the following equation. An average value of these values in three measurements was calculated, and evaluation was performed as follows.

Adsorbed amount (%) of lidocaine to the carrier film=[adsorbed amount of lidocaine to the carrier film/lidocaine content in the adhesive skin patch]×100

Determination ⊙: The adsorbed amount of lidocaine to the carrier film is less than 0.7%.

Determination ◯: The adsorbed amount of lidocaine to the carrier film is 0.7% or more and less than 1%.

TABLE 3

| | Carrier film | | Determination |
|---|---|---|---|
| | Material | Thickness (μm) | of adsorbed amount |
| Example 1 | hs-PET | 40 | ◯ |
| Example 2 | COC/PET | 45 | ⊚ |
| Example 3 | COC/PET/COC | 78 | ⊚ |

Test Result and Study thereto

As presented in Table 3, in Example 1, the adsorbed amount of lidocaine to the carrier film was 0.7% or more and less than 1%, which was favorable. In Example 2 and Example 3, the adsorbed amount of lidocaine to the carrier film was less than 0.7%, which was further favorable. From this result, it was considered that lidocaine is less likely to be adsorbed to the hs-PET film and further less likely to be adsorbed to the COC film.

### REFERENCE SIGNS LIST

1 Transdermal absorption preparation

2 Adhesive skin patch

3 Release liner

4 Adhesive layer

5 Backing film

6 Carrier film

1. A transdermal absorption preparation comprising an adhesive skin patch and a release liner, wherein
the adhesive skin patch includes
a backing film containing a knitted fabric and/or a nonwoven fabric,
an adhesive layer formed on one surface of the backing film and containing a pharmacologically active substance, and
a carrier film heat-sealed on a surface of the backing film opposite to a surface on which the adhesive layer is formed, and released after being applied to an injured part,
the release liner covers at least a part of the adhesive layer of the adhesive skin patch,
the carrier film has a bending resistance of 150 mm or less,
the backing film has a thickness of 100 μm to 1000 μm, and
the carrier film is a film having an area equal to or narrower than the backing film.

2. The transdermal absorption preparation according to claim 1, wherein a release force of the carrier film from the backing film is larger than a release force of the release liner from the adhesive layer of the adhesive skin patch.

3. The transdermal absorption preparation according to claim 1, wherein a release force of the carrier film from the backing film as measured at a release rate of 300 mm/min in a T-shape release test under conditions of 23° C. and 50 RH is 0.05 N/24 mm to 1 N/24 mm.

4. The transdermal absorption preparation according to claim 1, wherein the carrier film is formed from one or more of thermoplastic resin films.

5. The transdermal absorption preparation according to claim 1, wherein the carrier film is formed from one or more thermoplastic resin films selected from the group consisting of a cyclic olefin copolymer, polyethylene, polyethylene terephthalate, polypropylene, an ethylene vinyl alcohol copolymer, an ethylene vinyl acetate copolymer, polyvinylidene chloride, and polyacrylonitrile.

6. The transdermal absorption preparation according to claim 1, wherein the carrier film is formed from a film having a half-cut formed thereon.

7. The transdermal absorption preparation according to claim 1, wherein the carrier film is formed from a heat-sealable polyethylene terephthalate film.

8. The transdermal absorption preparation according to claim 1, wherein the carrier film is formed from a laminated body obtained by laminating a cyclic olefin copolymer, a polyethylene terephthalate film, and a cyclic olefin copolymer in this order.

9. The transdermal absorption preparation according to claim 1, wherein the carrier film is formed from a laminated body obtained by laminating a cyclic olefin copolymer and a polyethylene terephthalate film from the backing film side in this order.

10. The transdermal absorption preparation according to claim 1 being used for pasting to a shoulder, back, and/or waist.
